(19) United States
(12) Patent Application Publication
(10) Pub. No.: US 2005/0129728 A1
(43) Pub. Date: Jun. 16, 2005

(54) Sustained release pharmaceutical composition

(76) Inventors: Serge R. Martinod, Groton, CT (US); Malcolm Brandon, Bulleen (AU); Robert V. Packard, Wetherfield, CT (US)

Correspondence Address: MORGAN LEWIS & BOCKIUS LLP, 1111 PENNSYLVANIA AVENUE NW, WASHINGTON, DC, 20004, US

(21) Appl. No.: 10/943,947

(22) Filed: Sep. 20, 2004

Related U.S. Application Data
(63) Continuation-in-part of application No. 10/487,714, filed on Feb. 26, 2004.

(30) Foreign Application Priority Data
Sep. 11, 2001 (AU) PR 7614

(51) Int. Cl. A61F 2/00

(52) U.S. Cl. 424/423

(57) ABSTRACT

A sustained release mini-implant including a silicone support material; and a pharmaceutically active composition carried in or on the silicone support rod; the pharmaceutically active composition including at least one pharmaceutically active component; and optionally a carrier therefor; the mini-implant providing a predetermined threshold blood level of pharmaceutical active for treatment of a selected indication.

### CROSS REFERENCE TO RELATED APPLICATIONS

This application is a Continuation-in-Part of U.S. application Ser. No. 10/487,714, filed Feb. 26, 2004, which is a 371 National Phase filing of PCT/AU02/00868, filed Jul. 1, 2002, which claims priority to Australian Patent Application No. PR 7614, filed Sep. 11, 2001, all of which are incorporated by reference herein in their entirety.

The present invention relates to sustained release pharmaceutical compositions, and in particular a method for the preparation thereof. More specifically, the present invention relates to a sustained release pharmaceutical composition, which provides a significant increase in pharmaceutical payload.

A number of drug delivery systems are known in the prior art.

For example, a controlled drug-release preparation using as a carrier a hydrophobic polymer material, which is non-degradable after administration into the living body. There are two methods of controlling release of a drug from such preparation; one, using an additive such as an albumin (Japanese patent publication (Tokkohei) No. 61959/1995), and another, by forming an outer layer consisting of hydrophobic polymer alone (Japanese patent publication (Tokkaihei) No. 187994/1995).

However, where a disease indication requires the achievement of an initial high threshold blood plasma level and further requires the delivery of multiple pharmaceuticals and/or requires sustained release to subsequently be continued over an extended period at high levels, the drug delivery systems known in the prior art generally exhibit insufficient drug carrying capacity.

In addition, techniques known in the prior art for producing sustained release implants utilise a silicone based technology based on an extrusion or molding system.

Difficulties have been encountered in attempting to scale up such techniques to commercial volumes. Difficulties have also been encountered in applying such extrusion techniques to pharmaceutical actives such as Ceftiofur and Recombinant Porcine Somatotropin (rPST). For example, such activities interfere with silicone chemistry due to their chemical composition or exhibit temperature sensitivity.

It is, accordingly, an object of the present invention to overcome or at least alleviate one or more of the difficulties and deficiencies related to the prior art.

Accordingly, in a first aspect of the present invention, there is provided a sustained release mini-implant including
a silicone support material; and
a pharmaceutically active composition carried in or on the silicone support rod;
the pharmaceutically active composition including
   at least one pharmaceutically active component; and optionally a carrier therefor;
the mini-implant providing a predetermined threshold blood level of pharmaceutical active for treatment of a selected indication.

The sustained release mini-implant is preferably of the form of a matrix or an uncovered or covered rod. Whilst such apparatuses have been proposed in the prior art, such apparatuses in the prior art have been limited by their ability to provide relatively low loading capacities and/or their inability to deliver a zero order release profile optionally in combination with initial first order release.

Further, in International patent application PCT/AU02/00865, applicants disclose a sustained release apparatus including a plurality of sustained release mini-implants or pellets; each mini-implant including a sustained release support material; and a pharmaceutically active composition carried in or on the sustained release support material; the pharmaceutically active composition including at least one pharmaceutically active component; and a carrier therefor; each implant being of insufficient size and/or payload individually to provide a predetermined desired threshold blood level of pharmaceutical active for treatment of a selected indication; the size(s) of the mini-implants or pellets providing zero order release of pharmaceutical active; the sustained release apparatus providing, in use, zero order release of pharmaceutically active at, or above, the desired threshold level of pharmaceutical active for treatment of a selected indication.

Applicants have now surprisingly discovered that a single sustained release mini-implant according to the present invention may be used in place of the multiple implants required in the prior disclosure.

The sustained release mini-implant according to the present invention permits the treatment of diseases over an extended period with pharmaceutically active components which have heretofore not been applicable to such diseases as it has not been possible to achieve the required threshold blood plasma levels to be efficacious and to maintain those blood levels over an extended period of time.

The sustained release mini-implant may provide approximately zero order release of pharmaceutical active. Alternatively, where a rapid initial release of active is efficacious, the sustained release mini-implant may provide a hybrid first order/zero order release. That is release is initially rapid (first order) but quickly equilibrates to provide a generally constant low rate of release for an extended period.

The silicone support material may be formed from a silicone elastomer. The silicone support material may include a liquid silicone as described below.

The pharmaceutical carrier, when present, may include standard carrier components as described below.

The silicone support material may form a matrix or may exhibit a rod structure, preferably a covered rod structure, more preferably a co-extruded rod structure. A combination of a matrix implant and a covered rod implant may be used.

A partially covered rod may be used. Such a structure permits further modification of the release characteristics of the sustained release mini-implant according to the present invention. An eccentric or asymmetric rod, optionally partially or fully covered, may be used.

In a preferred aspect, the covered rod-type mini-implant according to the present invention provides approximately zero order release of pharmaceutical active.

In the sustained release mini-implant according to the present invention, the silicone support material may be formed from a silicone base polymer. The silicone base polymer may be of any suitable type. A biocompatible silicone base polymer is preferred. A methyl-vinyl polysiloxane polymer is preferred. A vinyl-substituted dimethyl siloxane polymer is particularly preferred. A low viscosity material is preferred, particularly for extrusion applications. A 40-durometer or lower formulation is preferred.

A reinforcing filler, e.g. a silica, preferably a fumed silica, may be included in the silicone base polymer. A silicone elastomer including fumed silica sold under the trade designations CS10401 or CS10701, and blends thereof, available from IMMIX Technologies LLC, Cri-Sil Division, have been found to be suitable. The reinforcing filler may be present in amounts of from approximately 1.0 to 33% by weight, preferably 10 to 20%, more preferably 10 to 15% by weight, based on the total weight of the sustained release mini-implant.

The silicone base polymer component may be present in amounts of from approximately 15 to 70% by weight, preferably approximately 25% to 65% by weight, based on the total weight of the mini-implant. The silicone base polymer can be either liquid form or “gum stock.” Preference is dictated by the type of process used to form and coat the sustained release mini-implant. Blending of multiple forms is a typical procedure for obtaining the desired physical properties.

The pharmaceutically active composition, as described above, may include
at least one pharmaceutically active component; and optionally a carrier therefor.

The pharmaceutically active component may include a water-insoluble pharmaceutical, a water-soluble pharmaceutical, a lipophilic pharmaceutical, or mixtures thereof.

The pharmaceutically active component may be exemplified by, but not limited to, one or more selected from the group consisting of:

| | |
|---|---|
| Acetonemia preparations | Anabolic agents |
| Anaesthetics | Analgesics |
| Anti-acid agents | Anti-arthritic agents |
| Antibodies | Anti-convulsivants |
| Anti-fungals | Anti-histamines |
| Anti-infectives | Anti-inflammatories |
| Anti-microbials | Anti-parasitic agents |
| Anti-protozoals | Anti-ulcer agents |
| Antiviral pharmaceuticals | Behaviour modification |
| | drugs |
| Biologicals | Blood and blood substitutes |
| Bronchodilators and expectorants | Cancer therapy and |
| | related pharmaceuticals |
| Cardiovascular pharmaceuticals | Central nervous system |
| | pharmaceuticals |
| Coccidiostats and coccidiocidals | Contraceptives |
| Contrast agents | Diabetes therapies |
| Diuretics | Fertility pharmaceuticals |
| Growth hormones | Growth promoters |
| Hematinics | Hemostatics |
| Hormone replacement therapies | Hormones and analogs |
| Immunostimulants | Minerals |
| Muscle relaxants | Natural products |
| Nutraceuticals and nutritionals | Obesity therapeutics |
| Ophthalmic pharmaceuticals | Osteoporosis drugs |
| Pain therapeutics | Peptides and polypeptides |
| Respiratory pharmaceuticals | Sedatives and tranquilizers |
| Transplantation products | Urinary acidifiers |
| Vaccines and adjuvants | Vitamins |

The pharmaceutically active component may include a water-insoluble pharmaceutical, a water-soluble pharmaceutical, a lipophilic pharmaceutical or mixtures thereof.

The water-soluble pharmaceuticals useful in the sustained release mini-implant according to the present invention include such drugs as peptides, polypeptides, proteins, glycoproteins, polysaccharides, and nucleic acids.

The present invention is particularly appropriate for pharmaceuticals that are very active even in extremely small quantities and whose sustained long-term administration is sought. When used in substantially increased quantities, such pharmaceuticals may be applied to disease indications heretofore untreatable over an extended period. The pharmaceuticals may be exemplified by, but not limited to, one or more selected from the group consisting of cytokines (eg. interferons and interleukins), hematopoietic factors (eg. colony-stimulating factors and erythropoietin (EPO)), hormones (eg. growth hormone, growth hormone releasing factor, calcitonin, leuteinizing hormone, leuteinizing hormone releasing hormone, and insulin), growth factors (eg. somatomedin, nerve growth factor), neurotrophic factors, fibroblast growth factor, and hepatocyte proliferation factor; cell adhesion factors; immunosuppressants; enzymes (eg. asparaginase, superoxide dismutase, tissue plasminogen activating factor, urokinase, and prourokinase), blood coagulating factors (eg. blood coagulating factor VIII), proteins involved in bone metabolism (eg. BMP (bone morphogenetic protein)), and antibodies (immunoglobulin (eg. gammaglobulin)).

Erythropoietin (EPO) and immunoglobulins are particularly preferred.

The interferons may include alpha, beta, gamma, or any other interferons or any combination thereof. Likewise, the interleukin may be IL-1, IL-2, IL-3, or any others, and the colony-stimulating factor may be multi-CSF (multipotential CSF), GM-CSF (granulocyte-macrophage CSF), G-CSF (granulocyte CSF), M-CSF (macrophage CSF), or any others.

Vaccines are particularly preferred. The vaccines useful in the sustained release mini-implant according to the present invention may be exemplified by, but not limited to, one or more selected from the group consisting of vaccines against

| | |
|---|---|
| Adenovirus | Anthrax |
| BCG | Chlamydia |
| Cholera | Circovirus |
| Classical swine fever | Coronavirus |
| Diphtheria-Tetanus (DT for children) | Diphtheria-Tetanus (tD for adults) |
| Distemper virus | DTaP |
| DTP | E coli |
| Eimeria (coccidosis) | Feline immunodeficiency virus |
| Feline leukemia virus | Foot and mouth disease |
| Hemophilus | Hepatitis A |
| Hepatitis B | Hepatitis B/Hib |
| Herpes virus | Hib |
| Influenza | Japanese Encephalitis |
| Lyme disease | Measles |
| Measles-Rubella | Meningococcal |
| MMR | Mumps |
| Mycoplasma | Para influenza virus |
| Parvovirus | Pasteurella |
| Pertussis | Pestivirus |
| Plague | Pneumococcal |
| Polio (IPV) | Polio (OPV) |
| Pseudorabies | Rabies |
| Respiratory syncitial virus | Rotavirus |
| Rubella | Salmonella |
| Tetanus | Typhoid |
| Varicella | Yellow Fever |

Pharmaceuticals that can be applied in pharmaceutically active compositions according to the present invention may be further exemplified by low-molecular-weight drugs such as water-soluble anticancer agents, antibiotics, anti-inflammatory drugs, alkylating agents, and immunosuppressants. Examples of these drugs include adriamycin, bleomycins, mitomycins, fluorouracil, peplomycin sulfate, daunorubicin hydrochloride, hydroxyurea, neocarzinostatin, sizofiran, estramustine phosphate sodium, carboplatin, beta-lactams, tetracyclines, aminoglycosides, and phosphomycin.

The pharmaceutically active composition of the present invention may contain two or more drugs depending on the disease and method of application.

Water-insoluble pharmaceutically active components which may be utilised in the sustained release mini-implant according to the present invention include lipophilic pharmaceuticals.

A lipophilic pharmaceutical may be any lipophilic substance so long as it is, as a form of a preparation, in a solid state at the body temperature of an animal or a human being to which the preparation is to be administered. Lipophilic as herein used means that the solubility of a substance in water is low, which specifically includes the following natures, as described in Pharmacopoeia of Japan 13th Edition (1996): practically insoluble (the amount of more than or equal to 10000 ml of solvent is required to dissolve 1 g or 1 ml of a solute), very hard to dissolve (the amount of more than or equal to 1000 ml and less than 10000 ml of solvent is required to dissolve 1 g or 1 ml of a solute), or hard to dissolve (the amount of more than or equal to 100 ml and less than 1000 ml of solvent is required to dissolve 1 g or 1 ml of a solute).

Specific examples of the lipophilic pharmaceutical include, but are not limited to, antibiotics such as avermectin, ivermectin, spiramycin, and ceftiofur; antimicrobials (eg. amoxicillin, erythromycin, oxytetracycline, and lincomycin), anti-inflammatory agents (eg. dexamethasone and phenylbutasone), hormones (eg. levothyroxine), adrenocorticosteroids (eg. dexamethasone palmitate, triamcinolone acetonide, and halopredone acetate), non-steroidal anti-inflammatory agents (eg. indomethacin and aspirin), therapeutic agents for arterial occlusion (eg. prostaglandin E1), anticancer drugs (eg. actinomycin and daunomycin), therapeutic agents for diabetes (eg. acetohexamide), and therapeutic agents for osteopathy (eg. estradiol).

Depending on a disease or a method for application, multiple lipophilic drugs may be contained. In addition to the lipophilic drug having a direct therapeutic effect, the drug may be a substance with a biological activity, and such a substance as promotes or induces a biological activity, which includes an adjuvant for a vaccine, for example saponin. In such a case, incorporation of a vaccine into a preparation results in a sustained release preparation of a vaccine with an adjuvant.

The pharmaceutically active composition may include an amount of pharmaceutical active component of approximately 15 to 85% by weight, preferably approximately 15 to 60% by weight, more preferably approximately 30 to 50% by weight, based on the total weight of the sustained release mini-implant.

As stated above, the pharmaceutically active composition according to the present invention may further include a carrier for the pharmaceutically active component.

The pharmaceutical carrier may be selected to permit release of the pharmaceutically active component over an extended period of time from the composition.

The carrier may include a water-soluble substance.

A water-soluble substance is a substance which plays a role of controlling infiltration of water into the inside of the drug dispersion. There is no restriction in terms of the water-soluble substance so long as it is in a solid state (as a form of a preparation) at the body temperature of an animal or human being to which it is to be administered, and a physiologically acceptable, water-soluble substance.

One water-soluble substance, or a combination of two or more water-soluble substances may be used. The water-soluble substance specifically may be selected from one or more of the group consisting of synthetic polymers (eg. polyethylene glycol, polyethylene polypropylene glycol), sugars (eg. sucrose, mannitol, glucose) sodium chondroitin sulfate, ammonium sulphate, polysaccharides (e.g. dextran, particularly dextran sulphate) amino acids (eg. glycine and alanine), mineral salts (eg. sodium chloride), organic salts (eg. sodium citrate or sodium glutamate) and proteins (eg. gelatin and collagen and mixtures thereof). A sugar or salt or mixtures thereof are preferred. A mixture of sodium chloride, sodium glutamate, ammonium sulphate and dextran sulphate is particularly preferred.

In addition, when the water-soluble substance is an amphipathic substance, which dissolves in both an organic solvent and water, it has an effect of controlling the release of, for example, a lipophilic drug by altering the solubility thereof. An amphipathic substance includes, but is not limited to, polyethylene glycol or a derivative thereof, polyoxyethylene polyoxypropylene glycol or a derivative thereof, a fatty acid ester, a sodium alkylsulfate of sugars, and more specifically, polyethylene glycol, polyoxy stearate 40, polyoxyethylene[196]polyoxypropylene-[67]glycol, polyoxyethylene[105]polyoxypropylene[5]glycol, polyoxyethylene[160]-polyoxypropylene[30]glycol, sucrose esters of fatty acids, sodium lauryl sulfate, sodium oleate, and sodium desoxycholic acid (sodium deoxycholic acid (DCA)).

Polyoxyethylene polyoxypropylene glycol (also called poloxymers as a generic term), sucrose, or a mixture of sucrose and sodium deoxycholic acid (DCA) are preferred.

In addition, the water-soluble substance may include a substance which is water-soluble and has any activity in vivo, such as low molecular weight drugs, peptides, proteins, glycoproteins, polysaccharides, or antigenic substances used as vaccines, i.e. water-soluble drugs.

The pharmaceutical carrier may constitute from 0% to approximately 50% by weight, preferably approximately 15% to 30% by weight, more preferably approximately 10% to 20% by weight, based on the total weight of the sustained release mini-implant.

The sustained release mini-implant may include additional carrier or excipients, fillers, lubricants, plasticisers, binding agents, pigments and stabilising agents.

Suitable fillers may be selected from the group consisting of talc, titanium dioxide, starch, kaolin, cellulose (microcrystalline or powdered) and mixtures thereof.

Where the sustained release mini-implant takes the form of a biocompatible article, e.g. an implant, calcium fillers, e.g. calcium phosphate, are particularly preferred.

Suitable binding agents include polyvinyl pyrrolidine, hydroxypropyl cellulose and hydroxypropyl methyl cellulose and mixtures thereof.

Accordingly, in a further aspect of the present invention, there is provided a process for the preparation of a sustained release mini-implant, which process includes
providing
   a silicone base polymer;
   a cross-linking agent;
   a pharmaceutically active component;
   a peroxide or metal catalyst; and
   a low temperature curing inhibitor;
pre-mixing at least a portion of the silicone base polymer and the metal catalyst together to form a first part;
pre-mixing the cross-linking agent, low temperature curing inhibitor, any remaining silicone base polymer, and pharmaceutical active for a time sufficient to at least partially wet the pharmaceutical active and form a second part; and
mixing the first and second parts together as a batch or continuously; and
feeding the mixture into a molding or extrusion apparatus at a relatively low temperature for a relatively short time sufficient to permit the components to cure to form the mini-implant.

It has surprisingly been found that the use of the process according to the present invention permits preparation of a sustained release mini-implant with significantly increased payloads.

As described above, the silicone base polymer may include a methyl-vinyl silicone polymer. The silicone base polymer may further include a reinforcing filler, e.g. a fumed silica. Fumed silica provides a high surface area relative to its weight so is preferred for high tear strength applications such as extrusion.

The process of preparing the sustained release mini-implant is a multi-step process; e.g. pre-mix, mix, form, cure, and optionally coat. This permits the composition to be mixed thoroughly with silicone base polymer before the pharmaceutical active and catalyst are brought into contact.

Accordingly, pharmaceutical actives, e.g. sulfur containing chemicals, which heretofore could not be used, e.g. due to inhibition of silicone curing, may be used in the process according to the present invention.

By utilising a pre-mixing step, potential interference between the pharmaceutical active and catalyst may be reduced or minimized. The pre-mixing process also enables more thorough dispersion of the pharmaceutical actives and carriers without adding to the “work-time” of the final silicone mixture.

Temperatures between approximately 100° C. to 200° C., preferably approximately 100° C. to 150° C. may be used.

As the process may be conducted at, or below, approximately 200° C., the method may be applied to the preparation of delivery systems for pharmaceutical actives including sensitive, particularly heat-sensitive, pharmaceutical actives. The duration of the curing step may range from approximately 30 seconds to 180 minutes depending upon the type of process used. For heat-sensitive actives, a curing time of approximately 30 seconds to 30 minutes at a temperature below the degradation temperature, preferably approximately 30 seconds to 15 minutes, more preferably approximately 45 seconds to 5 minutes, may be used.

The catalyst used may be a peroxide or metal catalyst. However, pharmaceutical actives, e.g. sulfur-containing pharmaceuticals, which heretofore could not be used, e.g. due to fouling of the catalyst, may be used in the process according to the present invention.

Such curing conditions are preferably achieved utilising a metal catalyst, more preferably a platinum or rhodium catalyst.

A platinum-containing catalyst is preferred for medical applications. If a platinum catalyst is used, it may or may not be attached to an organic ligand. The preferred catalyst is dependent upon the choice of inhibitor, concentration of inhibitor, concentration of cross-linker, and the desired curing profile.

Preferably the platinum catalyst is present in amounts of from approximately 0.05% to 0.25%, by weight, based on the total weight of the reaction mixture.

The relatively high concentration of metal catalyst may compensate for the relatively low temperatures at which the process is conducted. For convenience, the metal catalyst may be provided in a mixture with a portion of the silicone base polymer component.

As the process according to the present invention is conducted at such relatively low temperatures, a curing inhibitor that will act as a curing inhibitor at such low temperatures is required. Preferably the low temperature curing inhibitor includes an unsaturated cyclosiloxane, more preferably tetramethyl tetravinyl cyclosiloxane.

The amount of inhibitor used is dependent on the curing temperature selected, the lower the temperature the lower the concentration of inhibitor required. A concentration of approximately 2.5 to approximately 15% by weight preferably approximately 5 to 10% may be used.

In a preferred form, where the pharmaceutically active component does not tend to inhibit the silicone curing process, a portion of the pharmaceutically active component may be included in the first part. This is preferred where a high loading capacity of active is to be achieved.

In a preferred embodiment of the process of the present invention, a carrier for the pharmaceutical active may be included. Accordingly, the process may further include
providing a carrier for the pharmaceutically active component in an amount of from approximately 15% to 25% by weight based on the total weight of the reaction mixture; and
pre-mixing the pharmaceutical carrier in the first part.

The pharmaceutical carrier may preferably include a sodium chloride, mannitol or a mixture thereof.

Injection-molding processes may utilize up to 100% liquid silicone base polymer. Compression-molding or transfer-molding may utilise approximately 0.5 to 20% by weight, preferably approximately 2.5 to 7.5% by weight of a liquid silicone component. However an extrusion molding process, preferably a co-extrusion molding process, is preferred.

The cross-linking agent utilised in the process according to the present invention may be of any suitable type. A siloxane polymer; e.g. a partially methylated polysiloxane polymer, may be used. A short chain partially hydrogenated dimethyl siloxane polymer is particularly preferred.

The cross-linking agent may be present in amounts of from approximately 5% to 25% by weight, preferably approximately 10% to 15% by weight, based on the total weight of the reaction mixture.

As stated above, the sustained release mini-implant is preferably provided with a silicone coating. Accordingly in a preferred aspect of the present invention, the process may further include
providing a liquid coating composition; and
coating the apparatus with the coating composition.

The liquid coating composition may include a liquid silicone component, for example a liquid siloxane polymer.

The liquid coating composition may be applied utilising any standard technique. A dip coating process may be used, and the coating permitted to dry.

In a further preferred aspect of the present invention, the coating may be modified to provide a stronger coating layer and to extend the life of the implant. Accordingly, in this aspect, the process may further include
providing
   a liquid coating composition including
      a liquid silicone base material;
      a cross-linking agent; and
      metal catalyst
coating the apparatus with the coating composition; and
heating the coated apparatus to a temperature and for a time sufficient to cure the coating layer.

More preferably, the process may further include
providing a co-extrusion apparatus;
delivering the liquid coating composition to the co-extrusion apparatus; and
permitting the components to cure to form a co-extruded coated mini-implant such that the coating layer is delivered concentrically around the sustained release mini-implant.

The liquid silicone base material of the coating composition may be an unsaturated silicone, e.g. siloxane polymer. The liquid silicone base material may be the same as, or similar to, the low temperature curing inhibition material described above. A tetramethyl tetravinyl cyclosiloxane may be used.

The liquid silicone base material may be present in the coating composition in amounts of from approximately 35% to 95% by weight, preferably approximately 40% to 80% by weight, more preferably approximately 50% to 70% by weight, based on the total weight of the coating composition.

The cross-linking agent of the coating composition may be a short chain liquid siloxane polymer. The cross-linking agent may be the same as, or similar to, the cross-linking agent described above. A short chain hydrogenated dimethyl polysiloxane is preferred.

The metal catalyst may be a platinum or rhodium catalyst, as described above.

The coating process may be run utilising a batch process or may preferably be conducted continuously with the formation of the apparatus. For example the coating process may be conducted utilising a co-extrusion apparatus, such that the coating layer may be delivered concentrically around the sustained release mini-implant. The coating process may accordingly be conducted at temperatures and for times similar to those described above.

The cross-linking agent may be present in the coating composition in amounts of from approximately 2.5% to 25% by weight, preferably approximately 5% to 15% by weight, based on the total weight of the coating composition.

The sustained release mini-implant of the present invention may have a tablet or rod-like shape, for example it is selected from circular cylinders, prisms, and elliptical cylinders. When the device will be administered using an injector-type instrument, a circular cylindrical device is preferred since the injector body and the injection needle typically have a circular cylindrical shape, though other shaped objects may be used. For example, dog microchips may be administered using an injector type instrument.

The size of the pharmaceutical formulation of the present invention may, in the case of subcutaneous administration, be relatively small. For example using an injector-type instrument, the configuration may be circular cylindrical, and the cross-sectional diameter in this embodiment is preferably approximately 0.5 to 5.0 mm, more preferably 0.5 to 4 mm, and the axial length is preferably approximately 1 to 40 mm, more preferably 5 to 35 mm, most preferably 7.5 to 15 mm.

The thickness of the outer layer should be selected as a function of the material properties and the desired release rate. The outer layer thickness is preferably 0.02 mm to 2 mm, more preferably 0.10 mm to 1 mm, and even more preferably 0.15 mm to 0.2 mm.

The ratio of the axial length of the pharmaceutical formulation to the cross-sectional diameter of the inner layer may, in any case, be one or more and is more preferably two or more and most preferably five or more.

Where a double-layer structure is used, the pharmaceutical-containing inner layer and the drug-impermeable outer layer may be fabricated separately or simultaneously. Silicone is known for swelling with water and being gas-permeable.

A pharmaceutical formulation with an open end at one terminal may be fabricated by dipping one terminal of the pharmaceutical formulation into a solution which dissolves the outer-layer material and drying it, or by covering one terminal end of the pharmaceutical formulation with a cap made from the outer-layer material. In addition, the fabrication may comprise insertion of the inner layer into an outer-layer casing with a closed-end at one terminal, which are separately produced, and also formation of the inner layer in said casing.

In a further aspect of the present invention there is provided a method for the therapeutic or prophylactic treatment of a disease condition in an animal (including a human) requiring such treatment, which method includes administering to the animal a sustained release mini-implant including
a silicone support material; and
a pharmaceutically active composition carried in or on the silicone support material;
the pharmaceutically active composition including
   at least one pharmaceutically active component; and optionally
   a carrier therefor;
the mini-implant providing a predetermined threshold blood level of pharmaceutical active for treatment of a selected indication.

In a further preferred form, the method according to this aspect of the present invention permits the treatment, over an extended period, of diseases and related indications heretofore not treatable due to the required release profile of the pharmaceutical active.

In this form, the sustained release mini-implant may take the form of a biocompatible article as described above, e.g. medical apparatus or implant, as silicone support material.

In an alternative embodiment a hematopoietic factor (e.g. EPO) and/or antibody/immunoglobulin may be administered to an animal including a human. The required blood concentration may be maintained for an extended period.

Accordingly, in one embodiment, the present invention provides a method for the therapeutic or prophylactic treatment of irregularities in red blood cell production in an animal (including a human) requiring such treatment, which method includes administering to the animal a sustained release mini-implant including
a silicone support material; and
a pharmaceutically active composition carried in or on the silicone support material;
the pharmaceutically active composition including
   an erythropoietin (EPO) component; and optionally a carrier therefor;
the mini-implant providing a sustained release of EPO sufficient to promote a sustained increase in the level of circulating red blood cells.

Erythropoietin treatment may be indicated in the treatment of anemia associated with cancer chemotherapy, anemia associated with renal failure, rheumatoid arthritis, HIV infection, ulcerative colitis, and sickle cell anemia.

The method of administration may include subcutaneous or intramuscular injection, intranasal insertion or indwelling intrarectal insertion or indwelling, for example as a suppository or utilising oral administration.

The animals to be treated may be selected from the group consisting of sheep, cattle, goats, horses, camels, pigs, dogs, cats, ferrets, rabbits, marsupials, buffalos, yacks, primates, humans, birds including chickens, geese and turkeys, rodents including rats and mice, fish, reptiles and the like.

The method according to the present invention is particularly applicable to larger animals, e.g. cattle, sheep, pigs, dogs and humans where high dosage levels are required to achieve the prerequisite threshold pharmaceutical active blood levels for successful treatment of selected disease indications.

The present invention will now be more fully described with reference to the accompanying figures and examples. It should be understood, however, that the description following is illustrative only and should not be taken in any way as a restriction on the generality of the invention described above.

### EXAMPLE 1

Controlled Release of Human Immunoglobulin

Hybrid First Order/Zero Order Release

Formulation of Human Immunoglobulin (Gamma Globulin) for Controlled Release as a First Order/Zero Order Combination.

Implants formulated as a matrix and prepared as 3 mm diameter and 10 mm length. Human gamma globulin was incorporated into various formulations from 30-50% of final composition as shown in Table 1.

TABLE 1
Human Gamma Globulin Matrix in grams. Mini-extruder run
Matrix Formulation

| Implant | Gamma | | Dextran | MED- | CSM- | | | |
|---|---|---|---|---|---|---|---|---|
| Name | globulin | NaCl | Sulphate | 4104 | 4050-1 | PLY-7511 | CAT-55 | XL-112 |
| 2M | 2 | 0.25 | 0.5 | 1.5 | 0.25 | 0.093 | 0.004 | 0.006 |
| 3M | 2 | 0.75 | 0.25 | 1.5 | 0.5 | 0.093 | 0.004 | 0.006 |
| 4M | 1.5 | 1.5 | 0 | 1.5 | 0.5 | 0.093 | 0.004 | 0.006 |
| 5M | 2 | 1 | 0 | 1.5 | 0.5 | 0.093 | 0.004 | 0.006 |
| 6M | 2.5 | 0.5 | 0 | 1.5 | 0.5 | 0.093 | 0.004 | 0.006 |
| 7M | 2 | 0.5 | 0 | 1.5 | 0.5 | 0.093 | 0.004 | 0.006 |
| 8M | 3.0 | 0.5 | 0.5 | 1.5 | 0.5 | 0.093 | 0.004 | 0.006 |

M = Matrix type implant
Human gamma globulin was purchased from Sigma-Aldrich (St Louis USA) Catalogue No. G4388, 99% pure by electrophoresis.
MED 4104 silicone polymer (gum)
CSM 4050-1 silicone elastomer (base)
PLY-7511 dimethyl silicone polymer (fluid)
CAT-55 platinum catalyst
XL 112cross linker

Experimental Methods

The implants were placed into multiwell dishes using aseptic techniques. Each well contained 2 mls of phosphate buffered saline at pH 7.2 (PBS, pH 7.2). A single implant was placed into each well and the lid replaced. The multiwell dish was then kept in an incubator at 37° C. These conditions were used to mimic in vivo performance of the implants in human patients. The PBS, pH 7.2 was replaced at 9 am each day and the release of human IgG monitored using a BCA Protein Assay Reagent Kit (Number 23227) purchased from Pierce (Rockford, USA). The human IgG released from all the implants was confirmed to be undamaged from manufacture into implants and on prolonged incubation at 37° C. in having a molecular weight of 160,000 daltons by electrophoresis and standard gel chromatography techniques.

Results

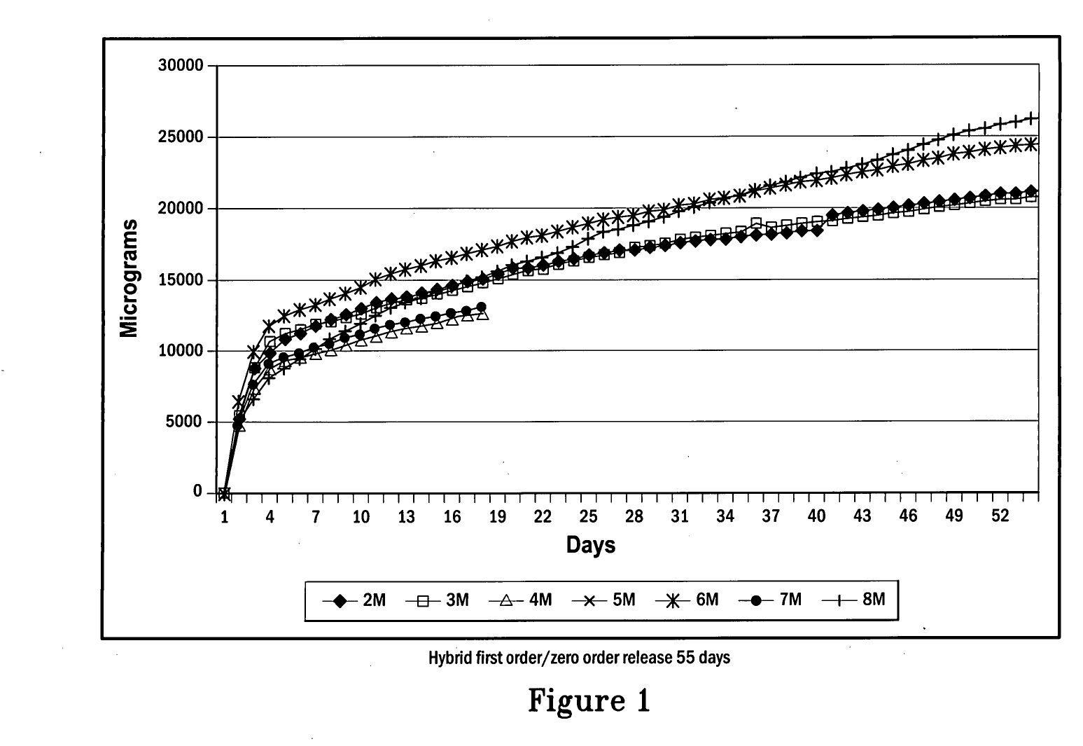

As shown in FIG. 1, the formulations prepared successfully allowed the controlled release of human gamma globulin at 37° C. for up to 55 days, at which point the experiment was stopped. It is highly likely controlled release of intact, unchanged human gamma globulin would have continued until the implant was exhausted of IgG.

Conclusions

1 It is possible to use “matrix” type single implants to achieve initial first order release followed by sustained zero order release. The most likely explanation for this pattern of release is that the initial “burst” release is a result of the human gamma globulin at the surface of the implant being released quickly which would achieve a high initial circulating level of immunoglobulin in the human patient, followed by a controlled release of the human gamma globulin in a zero order manner, reflecting a different mechanism of release from the silicone matrix after 4 days.
2 The in vitro results in physiological saline at pH 7.2 with human gamma globulin held at 37° C. for 55 days means that therapeutic human monoclonal antibodies could be formulated into the devices successfully and maintain their antibody properties without any deterioration of antibody titres over long periods.
3 Human monoclonal antibodies released from the hybrid first order/zero order matrix would allow for release of therapeutic monoclonal antibodies to achieve rapid therapeutic treatment in the first few days followed by the long term efficient maintenance of antibody levels in the human patient which would be far more efficacious and require only a single implantation of the patient.

### EXAMPLE 2

Controlled Release of Human Immunoglobulin

Zero Order Release

Formulation of Human Immunoglobulin (Gamma Globulin) for Zero Order Controlled Release.

Implants were formulated as a covered rod and prepared as 3 mm diameter and 10 mm length. Human gamma globulin was incorporated into various formulations from 30-50% of final composition as shown in Table 2.

TABLE 2
Human Gamma Globulin Covered Rod in Grams. Mini-extruder run

| | | | | | | | | | |
|---|---|---|---|---|---|---|---|---|---|
| | | Sodium | Ammonium | Dextran | | | | | |
| Ig G | NaCl | glutamate | Sulfate | Sulfate | MED-4104 | CSM-4050 | PLY 7511 | CAT-55 | XL-112 |

Covered Rod Formulation

| | | | | | | | | | | |
|---|---|---|---|---|---|---|---|---|---|---|
| 1 | 1.35 | 0.45 | 0 | 0 | 0 | 1.17 | 1.35 | 0.18 | 0.033 | 0.066 |
| 2 | 1.8 | 0.45 | 0 | 0 | 0 | 1.17 | 0.90 | 0.18 | 0.033 | 0.066 |
| 3 | 2.25 | 0.45 | 0 | 0 | 0 | 1.17 | 0.45 | 0.18 | 0.033 | 0.066 |
| 4 | 1.35 | 0.9 | 0 | 0 | 0 | 1.17 | 0.9 | 0.18 | 0.033 | 0.066 |
| 5 | 1.35 | 0 | 0.45 | 0 | 0 | 1.17 | 1.35 | 0.18 | 0.033 | 0.066 |
| 6 | 1.8 | 0 | 0.45 | 0 | 0 | 1.17 | 0.9 | 0.18 | 0.033 | 0.066 |
| 7 | 1.35 | 0 | 0.9 | 0 | 0 | 1.17 | 0.9 | 0.18 | 0.033 | 0.066 |
| 8 | 1.35 | 0 | 0 | 0.45 | 0 | 1.17 | 1.35 | 0.18 | 0.033 | 0.066 |
| 9 | 1.35 | 0.45 | 0 | 0.45 | 0 | 1.17 | 0.9 | 0.18 | 0.033 | 0.066 |
| C | 1.35 | 0 | 0 | 0 | 0 | 1.17 | 1.8 | 0.18 | 0.033 | 0.066 |
| 10 | 1.26 | 0.84 | 0 | 0 | 0 | 1.092 | 0.84 | 0.17 | 0.03 | 0.062 |
| 11 | 1.26 | 1.26 | 0 | 0 | 0 | 1.092 | 0.42 | 0.17 | 0.03 | 0.062 |
| 12 | 1.68 | 0.84 | 0 | 0 | 0 | 1.092 | 0.42 | 0.17 | 0.03 | 0.062 |
| 13 | 1.26 | 0.42 | 0 | 0 | 0.42 | 1.092 | 0.84 | 0.17 | 0.03 | 0.062 |

Matrix Formulation

| | | | | | | | | | | |
|---|---|---|---|---|---|---|---|---|---|---|
| 14M | 1.26 | 0.42 | 0 | 0 | 0.42 | 1.092 | 0.84 | 0.17 | 0.03 | 0.062 |

Experimental Methods

The experimental methods and reagents outlined for the hybrid first order/zero order formulations were used to test the zero order release of human gamma globulin formulations.

Results

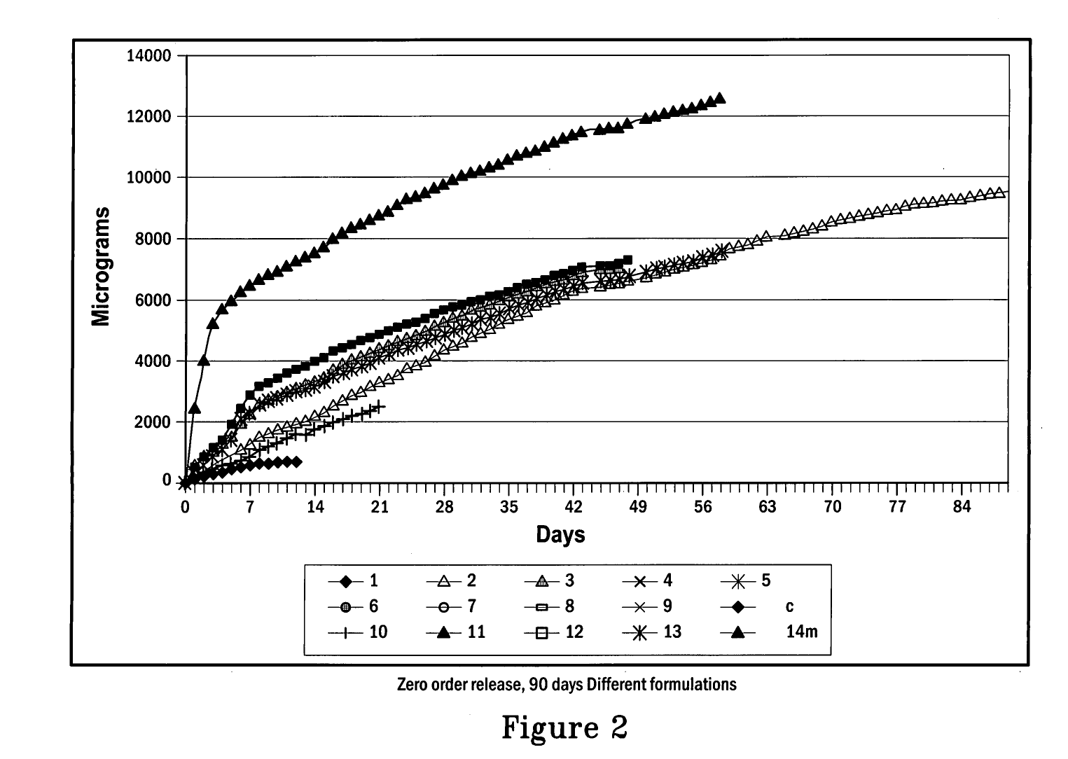

As can be observed in FIG. 2, the formulations successfully prepared allowed for the controlled release of human gamma globulin at 37° C. for up to 90 days at which time the experiment was stopped. For illustrative purposes a HYBRID formulation (14M) shows the contrast between first order and zero order release which was achieved using the covered rod implants. It is predicted that zero order controlled release of human gamma globulin would have continued until the implants were exhausted of human gamma globulin.

Conclusion

1 It is possible to use “covered rod” type single implants to achieve long term zero order release of human gamma globulin.
2 The in vitro results in physiological saline at pH 7.2 with human gamma globulin held at 37° C. for 90 days means that therapeutic human monoclonal antibodies could be formulated into implants and their stability maintained at 37° C. for 90 days to achieve maintenance of their antibody potency in human patients for at least 3 months. As the half life of human gamma globulin is around 20 days the controlled release of human monoclonal antibodies using both the matrix and covered rod types could be extended to 6-12 months using the formulations described.
3 Pegylation of human gamma globulin/monoclonal antibodies could also be used in conjunction with the implants for the long term release of human immunoglobulins.

### EXAMPLE 3

Recombinant Human EPO Implant Formulation

Recombinant human EPO was purchased from Ortho Biotech as 24 bottles of Procrit single dose preservative free vials, 1 ml each, containing 40,000 units each for a total of 1 million rhEPO units.

The contents of the 25 bottles were freeze dried to obtain 458 mg of a whole powder called EPO. The actual composition of the 458 mg of white powder contained according to the product insert
62.5 mg human albumin
144.5 mg NaCl
17.4 mg sodium citrate
1.5 mg citric acid
232.1 mg rhEPO
   Formulation of rhEPO as Covered Rod Silicone Implants

The rhEPO was formulated to achieve an actual 30% rhEPO covered rod implant.

The following ingredients were combined to produce covered rod implants:

| | |
|---|---|
| EPO white powder | 0.456 grams |
| MED 4104 | 0.276 grams |
| PLY-7511 | 0.0065 grams |
| XL 112 | 0.0135 grams |
| CAT-55 | 0.006 grams |
| MED 4104 | silicone polymer (gum) |
| CSM 4050-1 | silicone elastomer (base) |
| PLY-7511 | dimethyl silicone polymer (fluid) |
| CAT-55 | platinum catalyst |
| XL 112 | cross linker |

Experimental Protocol
Sponsor:
   Smart Drug Systems Inc
Study Investigator:
   CPC Veterinary Consulting Pty Ltd
   3/206 Tucker Rd
   Bentleigh Vic Australia, 3206.
   Craig Cunningham BVSc PhD
Study Location:
   ACA Breeders Kennels
   RMB 1492
   South Gippsland Highway
   Longford Vic Australia
      Purpose:

To determine the feasibility of using a single silicone implant containing human recombinant erythropoietin (rhEPO) to promote a sustained increase in the level of circulating red blood cells (packed cell volume—PCV).

Implants:

Each dog was given a single silicone implant 1.5 mm×10 mm.

Dogs:

4 female beagles—1 control and 3 implanted with a single silicone implant.

Dosage:

Dogs were implanted with a single implant containing the equivalent amount of rhEPO for conventional use in dogs for 6 weeks (3 injections first week, 2 injections per week for next 5 weeks).

Procedure:

Dogs (3) were implanted subcutaneously with silicone implant containing EPO. A blood sample (EDTA—whole blood) was collected prior to implanting for baseline PCV measurement. PCV was measured using VetTest haemogram machine at Moorabbin Veterinary Hospital. Dogs were blood sampled weekly for 4 weeks.

TABLE 3

| Dog No | 0 | 1 | 2 | 3 | 4 |
|---|---|---|---|---|---|
| | Packed Cell Volume (PCV) | | | | |
| | Weeks Post-Implantation | | | | |
| 1 | 50.3 | 56.6 | 62.1 | 57.6 | 52.3 |
| 2 | 49.6 | 56.9 | 56.2 | 55.5 | 49.9 |
| 3 | 49.6 | 54.1 | 54.7 | 52.5 | 45.5 |
| 4* | 45.3 | 43.0 | 39.1 | 43.0 | 38.4 |
| | Percentage Change in Packed Cell Volume | | | | |
| | Weeks Post-Implantation | | | | |
| 1 | — | +12.5 | +24 | +14.5 | +4 |
| 2 | — | +14.7 | +13 | +11.9 | +0.6 |
| 3 | — | +9 | +10 | +5.9 | −8 |
| 4* | — | −5 | −13 | −5.1 | −15 |

*Control dog - not treated with EPO.

Conclusions
1 Controlled release of rhEPO was successfully achieved for 3 weeks as clearly shown in Table 3.
2 The successful elevation of packed cell volume in dogs for 3 weeks shows that the formulation and manufacture of the silicone implant did not affect the biological potency of rhEPO.
3 It is likely the release rate of rhEPO was too fast, as a result of the high salt concentration used by the rhEPO manufacturer. Lowering of the salt concentration would lead to a significantly longer zero order release profile for rhEPO.
4 Other techniques including pegylation that prolong the half life of rhEPO would also lead to increased efficiency using the silicone implant.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and may be used interchangeably and should not be taken as excluding the presence of other elements or features.

1. A sustained release mini-implant including
a silicone support material; and
a pharmaceutically active composition carried in or on the silicone support rod;
the pharmaceutically active composition including
at least one pharmaceutically active component; and optionally a carrier therefor;
the mini-implant providing a predetermined threshold blood level of pharmaceutical active for treatment of a selected indication.

2. A sustained release mini-implant according to claim 1, wherein the mini-implant provides approximately zero order release of pharmaceutical active.

3. A sustained release mini-implant according to claim 2, wherein the mini-implant has a covered rod structure.

4. A sustained release mini-implant according to claim 3, wherein the silicone support rod has a molded or extruded rod structure.

5. A sustained release mini-implant according to claim 4, wherein the silicone support rod has a coated rod structure.

6. A sustained release mini-implant according to claim 5, wherein the silicone support rod has a co-extruded rod structure.

7. A sustained release mini-implant according to claim 1, wherein the mini-implant has a matrix-type structure.

8. A sustained release mini-implant according to claim 7, wherein the mini-implant provides a hybrid first order/zero order release of pharmaceutical.

9. A sustained release mini-implant according to claim 1, wherein the silicone support material is formed from a silicone base polymer including a methyl-vinyl polysiloxane polymer.

10. A sustained release mini-implant according to claim 9, wherein the silicone base polymer includes a silicone elastomer including a fumed silica as reinforcing filler.

11. A sustained release mini-implant according to claim 10, wherein the silicone base polymer is present in amounts of from approximately 15% to 70% by weight, based on the total weight of the apparatus.

12. A sustained release mini-implant according to claim 1, wherein the pharmaceutically active composition includes a pharmaceutically active component selected from one or more of the group consisting of acetonemia preparations, anabolic agents, anaesthetics, analgesics, anti-acid agents, anti-arthritic agents, antibodies, anti-convulsivants, anti-fungals, anti-histamines, anti-infectives, anti-inflammatories, anti-microbials, anti-parasitic agents, anti-protozoals, anti-ulcer agents, antiviral pharmaceuticals, behaviour modification drugs, biologicals, blood and blood substitutes, bronchodilators and expectorants, cancer therapy and related pharmaceuticals, cardiovascular pharmaceuticals, central nervous system pharmaceuticals, coccidiostats and coccidiocidals, contraceptives, contrast agents, diabetes therapies, diuretics, fertility pharmaceuticals, growth hormones, growth promoters, hematopoietic factors, hematinics, hemostatics, hormone replacement therapies, hormones and analogs, immunostimulants, minerals, muscle relaxants, natural products, nutraceuticals and nutritionals, obesity therapeutics, ophthalmic pharmaceuticals, osteoporosis drugs, pain therapeutics, peptides and polypeptides, respiratory pharmaceuticals, sedatives and tranquilizers, transplantation products, urinary acidifiers, vaccines and adjuvants and vitamins.

13. A sustained release mini-implant according to claim 12, wherein the pharmaceutical active is selected from one or more of the group consisting of hematopoietic factors and antibodies.

14. A sustained release mini-implant according to claim 13, wherein the pharmaceutical active includes erythropoietin or human immunoglobulin.

15. A sustained release mini-implant according to claim 14, wherein the pharmaceutical active further includes at least one pharmaceutically active component selected from the group consisting of acetonemia preparations, anabolic agents, anaesthetics, analgesics, anti-acid agents, anti-arthritic agents, antibodies, anti-convulsivants, anti-fungals, anti-histamines, anti-infectives, anti-inflammatories, anti-microbials, anti-parasitic agents, anti-protozoals, anti-ulcer agents, antiviral pharmaceuticals, behaviour modification drugs, biologicals, blood and blood substitutes, bronchodilators and expectorants, cancer therapy and related pharmaceuticals, cardiovascular pharmaceuticals, central nervous system pharmaceuticals, coccidiostats and coccidiocidals, contraceptives, contrast agents, diabetes therapies, diuretics, fertility pharmaceuticals, hematinics, hemostatics, hormone replacement therapies, hormones and analogs, immunostimulants, minerals, muscle relaxants, natural products, nutraceuticals and nutritionals, obesity therapeutics, ophthalmic pharmaceuticals, osteoporosis drugs, pain therapeutics, peptides and polypeptides, respiratory pharmaceuticals, sedatives and tranquilizers, transplantation products, urinary acidifiers, vaccines and adjuvants and vitamins.

16. A sustained release mini-implant according to claim 14, wherein the pharmaceutical active further includes a vaccine component selected from one or more of the group consisting of vaccines against Adenovirus, Anthrax, BCG, Chlamydia, Cholera, Circovirus, Classical swine fever, Coronavirus, Diphtheria-Tetanus, Distemper virus, DTaP, DTP, E coli, Eimeria (coccidosis), Feline immunodeficiency virus, Feline leukemia virus, Foot and mouth disease, Hemophilus, Hepatitis A, Hepatitis B, Hepatitis B/Hib, Herpes virus, Hib, Influenza, Japanese Encephalitis, Lyme disease, Measles, Measles-Rubella, Meningococcal, MMR, Mumps, Mycoplasma, Para influenza virus, Parvovirus, Pasteurella, Pertussis, Pestivirus, Plague, Pneumococcal, Polio (IPV), Polio (OPV), Pseudorabies, Rabies, Respiratory syncitial virus, Rotavirus, Rubella, Salmonella, Tetanus, Typhoid, Varicella and Yellow Fever.

17. A process for the preparation of a sustained release mini-implant, which process includes
providing
a silicone base polymer;
a cross-linking agent;
a pharmaceutically active component;
a peroxide or metal catalyst; and
a low temperature curing inhibitor;
pre-mixing at least a portion of the silicone base polymer and the metal catalyst together to form a first part;
pre-mixing the cross-linking agent, low temperature curing inhibitor, any remaining silicone base polymer, and pharmaceutical active for a time sufficient to at least partially wet the pharmaceutical active and form a second part; and
mixing the first and second parts together as a batch or continuously; and
feeding the mixture into a molding or extrusion apparatus at a relatively low temperature for a relatively short time sufficient to permit the components to cure to form the mini-implant.

18. A process according to claim 17, wherein the silicone base polymer includes a methyl-vinyl siloxane polymer.

19. A process according to claim 17, wherein the silicone base polymer further includes a reinforcing filler.

20. A process according to claim 17, wherein the cross-linking agent includes a partially hydrogenated polysiloxane polymer.

21. A process according to claim 17, wherein the catalyst is a platinum catalyst present in amounts of from approximately 0.05% to 0.25%, by weight, based on the total weight of the reaction mixture.

22. A process according to claim 17, wherein the low temperature curing inhibitor includes a tetramethyl tetra-vinyl cyclosiloxane.

23. A process according to claim 17, further including
providing a carrier for the pharmaceutically active component in an amount of from approximately 15% to 25% by weight based on the total weight of the reaction mixture; and
pre-mixing the pharmaceutical carrier in the first part.

24. A process according to claim 23, wherein the pharmaceutical carrier includes sodium chloride, sodium glutamate, ammonium sulphate, dextran sulphate or mixtures thereof.

25. A process according to claim 23, wherein a portion of the pharmaceutically active component is included in the first part.

26. A process according to claim 17, further including
providing
a liquid coating composition including
a liquid silicone base material;
a cross-linking agent; and
metal catalyst
coating the apparatus with the coating composition; and
heating the coated apparatus to a temperature and for a time sufficient to cure the coating layer.

27. A process according to claim 26, further including
providing a co-extrusion apparatus;
delivering the liquid coating composition to the co-extrusion apparatus; and
permitting the components to cure to form a co-extruded coated mini-implant such that the coating layer is delivered concentrically around the sustained release mini-implant.

28. A mini-implant produced by a process according to claim 17.

29. A method for the therapeutic or prophylactic treatment of a disease condition in an animal (including a human) requiring such treatment, which method includes administering to the animal a sustained release mini-implant including
a silicone support material; and
a pharmaceutically active composition carried in or on the silicone support rod;
the pharmaceutically active composition including
at least one pharmaceutically active component; and optionally a carrier therefor;
the mini-implant carrying sufficient payload to provide a predetermined threshold blood level of pharmaceutical active for treatment of a selected indication.

30. A method according to claim 1, wherein the pharmaceutically active component includes a hematopoietic factor or antibody source.

31. A method for the therapeutic or prophylactic treatment of irregularities in red blood cell production in an animal (including a human) requiring such treatment, which method includes administering to the animal a sustained release mini-implant including
a silicone support material; and
a pharmaceutically active composition carried in or on the silicone support material;
the pharmaceutically active composition including
an erythropoietin (EPO) component; and optionally a carrier therefor;
the mini-implant providing a sustained release of EPO sufficient to promote a sustained increase in the level of circulating red blood cells.
